(19) United States
(12) Patent Application Publication
(10) Pub. No.: US 2008/0132475 A1
(43) Pub. Date: Jun. 5, 2008

(54) Treatment for dry eye

(76) Inventors: Charles Gerald Connor, Germantown, TN (US); Charles L. Haine, Memphis, TN (US)

Correspondence Address: REED SMITH LLP, 2500 ONE LIBERTY PLACE, 1650 MARKET STREET, PHILADELPHIA, PA, 19103, US

(21) Appl. No.: 11/634,347

(22) Filed: Dec. 5, 2006

(51) Int. Cl. A61K 31/57

(52) U.S. Cl. 514/178; 514/177

(57) ABSTRACT

The present invention comprises a composition and method treating eye diseases using a composition having a therapeutically effective amount of a progestagen and a pharmaceutically acceptable carrier, wherein the composition is applied to the palpebral part of the eye and/or ocular surface.

### TECHNICAL FIELD OF THE INVENTION

The invention generally relates to compositions and methods for treating eye diseases, in particular dry eye with progestagens, wherein the composition is applied to the palpebral part of the eye and/or the ocular surface.

### BACKGROUND OF THE INVENTION

Dry eye, also known as Keratoconjunctivitis Sicca (“KCS”), is a condition in which the quality and/or quantity of tears bathing the eye decline. People who have dry eye may experience inflammation, dryness and/or foreign body sensation in the conjunctival region of the eye, light sensitivity, itching, burning or stinging, grittiness, tired eyes, contact lens intolerance, and blurring of vision. Almost all dry eye disorders a result of a loss of water from the tear film. The loss of water from the tear film may be caused by a decrease in tear production and/or an increase in evaporation of tears, which may be a result of an abnormality in mucin or lipid components of the tear film. These phenomena may occur together, but both typically result in increased osmolarity from the normal limit of 311 mOsm/L and may ultimately lead to a decrease in goblet-cell density. A decrease in goblet-cell density affects the production of mucus, which is the major lubricant in the tear film. This aggravates and/or causes inflammation by T-cell activation, causing inflammatory cytokines to be released.

It has also been shown that patients with chronic dry eye experience increased activation of T-cells. These T-cells release cytokines that may result in: (1) neural signals to the lacrimal gland that disrupt production of natural tears leading to a decrease tear production; (2) tissue damage in the lacrimal glands and/or ocular surface; (3) recruitment of additional T-cells; and/or (4) increased inflammatory cytokine production.

Conditions that may give rise to dry eye include, but are not limited to Sjögren syndrome, blepharitis, meibomian gland disorder, HIV, herpes zoster, autoimmune disease, the natural aging process, diabetes, long-term contact lens wear, dry environment, surgery that involves corneal incisions or ablates corneal nerves, medications, decreased blinking, eyelids that cannot be closed, pregnancy, polycystic ovary syndrome, acne rosacea, Lupus, Scleradenma, Sarcoidosis, Stevens-Johnson syndrome, Parkinson's, smoking, radiation therapy, vitamin A deficiency, and menopause. This wide divergence in causative factors makes it particularly difficult to fashion a successful treatment for dry eye.

Generally, the tear film is made up of three layers: (I) an innermost hydrophilic mucin layer produced by the conjunctiva goblet cells and the ocular surface epithelium and which serves as an anchor for the tear film, helping it adhere to the eye; (2) a middle thick aqueous layer produced by the lacrimal glands; and (3) a superficial thin lipid layer produced by the meibomian glands, which helps with uniform tear spreading and to slow down tear evaporation. This three-layer structure stabilizes the tear film and enables the tear film to keep the eye moist, create a smooth surface for light to pass through the eye, nourish the front of the eye, and provide protection from injury and infection. The quality of tears in a dry eye sufferer is typically deficient with respect to this protective and stabilizing structure.

There are several techniques for diagnosing and evaluating the severity of a patient's dry eye, including the Ocular Surface Disease Index (OSDI) questionnaire, Tear Break-up Time, tear staining, tear film height, and the Schirmer Test. See Milder, B, The Lacrimal System, Appleton-Century-Crofts, Chapter 8, 1993 and Schirmer, 0 Studien Zur Physiologie and Pathologie der Tranenabsonderdung und Tranenabfuh, Arch kiln ophthalmol, 1903; 56:197-291, each of which is herein incorporated by reference in its entirety including any references cited therein. Each test provides different information about the tear film of a patient.

The patient's subjective evaluation of the severity of the symptoms can be recorded using the standardized OSDI questionnaire. This subjective evaluation can be confirmed by objective indicators such as the Tear Break-up Time (TBUT) test, and the Schirmer Test. The TBUT test measures the time required for the three-layer tear film to break up. A shortened TBUT test time indicates a decreased quality of tears and is indicative of dry eye. See Lemp et al., Factors Affecting Tear Film Break Up in Normal Eyes, Arch Ophthalmol 1973; 89:103-105, which is herein incorporated by reference in its entirety including any references cited therein. The Schirmer Test measures the volume of tears produced, and is performed by of placing a small strip of filter paper inside the lower eyelid (conjunctival sac) of each eye for several minutes, allowing tear fluid to be drawn into the filter paper by capillary action. The paper is then removed and the amount of moisture is measured in millimeters. Typically, a measurement of less than 5 mm indicates dry eye. Schirmer, 0 Studien zur physiologie and pathologie der tranenabsonderdung und tranenabfuh, Arch kiln ophthalmol, 1903; 56:197-291.

Current treatments for dry eye include artificial tears, such as ointments and gels for application to the ocular surface. These provide basic lubrication to the eye surface. Restasis® eye drops (cyclosporine in a castor oil base) are said to help the eyes increase tear production. Other treatments include temporary and permanent punctal occlusions, topical androgen eye drops, topical antibiotics, and oral therapy with polyunsaturated fatty acids. For example, U.S. Pat. No. 6,659,985, herein incorporated by reference in its entirety including any references cited therein, discloses using androgens for the treatment of dry eye by applying the composition to the adnexa of the eye.

There are draw backs to the current dry eye treatments. For example, Restasis® is said to have a slow onset of action, appears to help only about 20% of patients, does not appear to work for severe dry eye cases, and has side effects such as burning on instillation. With punctal plugs infection may occur and surgical removal may be required. Topical administration of steroids may have such adverse effects as increase in intraocular pressure, glaucoma, cataract, and exacerbation of corneal infection. See Butcher, et al., Bilateral Cataracts and Glaucoma Induced by Long Term Use of Steroid Eye Drops, BMJ, 1994; 309:43, which is herein incorporated by reference in its entirety including any references cited therein.

There are three types of sex steroid hormones: androgens, estrogens, and progestagens. The progestagens are hormones which have progestational activity, i.e. produce effects similar to progesterone (the only natural progestagen), such as preparing the uterus for the reception and development of the fertilized ovum by transforming the endometrium from the proliferative to the secretory stage and maintaining an optimal intrauterine environment for sustaining pregnancy. As referred to herein, the term “progestagen” includes but is not limited to progesterone, synthetic progestagens (which are sometimes referred to in the art as “progestins”), medroxyprogesterone acetate (medrysone), norethindrone (or norethisterone), norethindrone acetate, megestrol acetate, 17-a-hydroxyprogesterone caproate, and norgestrel, and derivatives thereof. Natural progesterone does not have any serious clinical side effects nor have any toxic levels been identified.

Progesterone is both a final product of the steroid hormone pathway as well as an intermediate in the synthesis of cortisol. This pathway occurs in both men and women. In women, progesterone is produced in the corpus luteum of the ovary as well as the placenta and adrenal cortex in both sexes. Progesterone, in contrast to estrogen, is mildly catabolic in humans and can be thought of as balancing the action of estrogen. The biological actions of progesterone are diverse and often opposing. Its effect on target tissues is mediated by progesterone receptors that function as ligand-activated transcription factors to regulate the expression of specific sets of target genes. The progesterone receptor belongs to a large family of nuclear receptors which include receptors for the following: (i) steroid hormones (estrogen, progesterone, glucocorticoid, androgen, and mineralcorticoid); (ii) other lipophilic hormones and ligands (thyroid hormone, retinoic acid, 9-cis retinoic acid, vitamin D₃, eicosanoids, fatty acids, and lipids); and (iii) orphan receptors that have no known ligand. The progesterone receptor and corticosteroid receptor share regions of high homology, particularly within the DNA-binding domain of the steroid hormone receptor family which results in cross reactivity. The precise physiological effects of progestagens can be difficult to interpret due to their potential to cross-react with other nuclear receptors, such as glucocorticoid, mineralcorticoid, and androgen receptors.

Progestagens may have cross-reactivity with other sex hormones such as by acting on different types of receptors, but with respect to the present invention, progestagens are those molecules that predominantly have progestational activity.

Progestagens are currently used: (1) in the prevention of miscarriage; (2) to treat various cancers, such as breast, kidney, and uterine; (3) to treat menstrual disorders and other gynecological disorders; (4) as an oral contraceptive; (5) in hormone replacement therapy (HRT); (6) to treat loss of appetite and severe weight and/or muscle loss due to AIDS and/or cancer; and (7) as an antiandrogen. In the treatment of these disorders progestagens are used in many forms such as pills, injections, vaginal suppositories, and skin creams. However, up until the present invention, progestagens have not been used to treat dry eye. Further, no treatment currently exists where a composition having at least one progestagen is applied to the palpebral part of an eye and/or the ocular surface to treat dry eye. As referred to in the instant specification, the term “palpebral part of an eye” is the external portion of the upper and lower eyelids and the medial and lateral canthus.

### SUMMARY OF THE INVENTION

The present invention relates to compositions and methods for treating eye disease, and in particular dry eye wherein the composition has a therapeutically effective amount of a progestagen and a pharmaceutically acceptable carrier. The compositions may be applied to the palpebral part of the eye, which includes the upper and lower eyelids and the medial and lateral canthus and/or may be applied to the ocular surface, which includes the conjuntiva. Further, the invention relates to compositions and methods for treating dry eye wherein the composition has a therapeutically effective amount of progesterone. The amount of progestagen will vary based upon the desired treatment amount, severity of the eye disease, and carrier used in the formulation of the composition. Further, the pharmaceutically acceptable carrier may include any carrier known in the art for use with topical application to the skin and transdermal delivery of a sex steroid hormone, or which is known to be suitable for delivery to the conjunctiva.

### DETAILED DESCRIPTION

Reference will now be made in detail to embodiments of the invention. While the invention will be described in conjunction with the embodiments, it will be understood that they are not intended to limit the invention to those embodiments. On the contrary, the invention is intended to cover alternatives, modifications, and equivalents, which may be included within the spirit and scope of the invention as defined by the appended claims.

The present invention relates to compositions and methods for treating dry eye using at least one progestagen.

The present invention includes a composition having a therapeutically effective amount of a progestagen and a pharmaceutically acceptable carrier. According to a preferred embodiment, the composition has a therapeutically effective amount of progesterone and a pharmaceutically acceptable carrier. Three forms of progesterone are recognized by the U.S. Pharmacopoeia, namely progesterone USP micronized, progesterone USP wettable microcrystalline, and progesterone USP milled. Each of these forms may be used with the present invention, preferably progesterone USP milled.

When applied to the palpebral region, the amount of progestagen in the composition may range from about 2% to about 30%, preferably from about 10% to about 30%, more preferably from about 15% to about 25%, and even more preferably about 15%. The amount of progesterone in the composition may range from about 2% to about 30%, preferably from about 10% to about 30%, more preferably from about 15% to about 25%, and even more preferably about 15%. The amount of progestagen in the composition will vary depending upon the pharmaceutically acceptable carrier used and the desired concentration delivered to a patient for treatment.

The present invention may be applied once a day or more depending upon, but not limited to, the needs of the patient and/or the severity of the condition. Preferably, it is applied twice a day. The amount of the progestagen composition that is applied to each eye per day will vary depending on, but not limited to, the severity of the dry eye and/or number of applications and may range from about 25 mg to about 500 mg, preferably from about 100 mg to about 400 mg, more preferably about 160 mg.

Pharmaceutically acceptable carriers for use with the formulations of the present invention are well known in the cosmetic and pharmaceutical arts, and include but are not limited to such vehicles (or vehicle components) as water; organic solvents such as alcohols (particularly lower alcohols readily capable of evaporating from the skin such as ethanol), glycols (such as glycerin), aliphatic alcohols; mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes, protein-based materials such as collagen and gelatin; silicon-based materials (both non-volatile and volatile) such as cyclomethicone, demethiconol and dimethicone copolyol (Dow Corning); hydrocarbon-based materials such as petrolatum and squalane; anionic, cationic and amphoteric surfactants; sustained-release vehicles such as microsponges and polymer matrices; stabilizing and suspending agents; emulsifying agents, and other vehicles and vehicle components that are suitable for administration to the skin, as well as mixtures of topical vehicle components as identified above or otherwise known to the art.

The pharmaceutically acceptable carrier may further include components adapted to improve the stability or effectiveness of the applied formulation, such as preservatives, antioxidants, skin penetration enhancers, sustained release materials, and the like. Examples of such vehicles and vehicle components are well known in the art.

The composition may be in the form a gel, a cream, a lotion, a solution, or an ointment.

The pharmaceutically acceptable carrier may also be a commercially available neutral base known in the art. A neutral base has no significant therapeutic effect of its own. It simply conveys the active pharmaceutical ingredient, although some vehicles may do so with greater ease or effectiveness than others. A neutral base may be a cream used cosmetically for softening and/or cleaning the skin. Examples include Eucerin® (Beiersdorf Aktiengesellschaft Corp., Hamburg, Germany), Aquaphor® (Beiersdorf Aktiengesellschaft Corp., Hamburg, Germany), and liposomal vehicles. A preferred neutral base is Vanicream® (Pharmaceutical Specialties, Inc., Rochester, Minn.). Vanicream® is composed of purified water, white petrolatum, cetearyl alcohol and ceteareth-20, sorbitol solution, propylene glycol, simethicone, glyceryl monostearate, polyethylene glycol monostearate, sorbic acid and butylated hydroxytoluene (BHT).

The pharmaceutically acceptable carrier may be a transdermal gel such as Pluronic Lecithin Organogel (PLO). See Murdan, A Review of Pluronic Lecithin Organogel as a Topical and Transdermal Drug Delivery System, Hospital Pharmacist, July/August 2005, Vol. 12, pp. 267-270, which is herein incorporated by reference in its entirety including any references cited therein.

One or more penetration enhancers may be included in the composition of the present invention. The types of penetration enhancers include, but are not limited to, phospholipids, terpenes, anionic surfactants, cationic surfactants, zwitterionic surfactants, nonionic surfactants, fatty acids, fatty esters, fatty amines, azone-like compounds, and sodium salts of fatty acids.

The topical application of a progestagen composition of the present invention to the palpebral part of the eye allows for easy application and for transdermal delivery of the active ingredient to the sites of action, which may include but is not limited to the ocular surface, which includes the cornea and conjunctiva; the lacrimal gland and lacrimal accessory glands; and the meibomian glands. This form of transdermal delivery provides effective treatment without the side effects caused by systemic use of the drug. These side effects of oral progesterone include, but are not limited to upset stomach, cramps, breast tenderness, drowsiness, dizziness, headache, migraine headache, vomiting, diarrhea, constipation, tiredness, skin rash, and lower levels of high density lipoprotein (HDL).

The topical composition of the present invention may also be applied to the ocular surface (as distinguished from the palpebral region), which includes the cornea and conjunctiva. In this case, the composition is typically in the form of drops or an ointment. The amount of progestagen in a composition for application to the ocular surface may range from about 0.1% to about 10%, preferably about 2%.

The topical application of the present invention to the ocular surface may be applied once a day or more frequently based upon, but not limited to, the needs of the patient and/or the severity of the condition. Preferably, it is applied between about 4 times to about 8 times per day. A few drops of the progestagen composition may be applied to the ocular surface as needed for each application.

Transdermal delivery and topical application of the progestagen composition will have little or no systemic side effects typically caused by oral use and/or injection of steroid hormones. The concentration of the progestagen composition is high enough to affect the area to which the composition is applied as well as the targeted structures of the eye, but low enough to prevent the typical side effects associated with systemic hormone treatments.

The present invention may also be used in combination with other skin treatment ingredients, such as but not limited to sunscreen, vitamins, plant extracts, and moisturizers.

The progestagen may be prepared for inclusion in the composition of the present invention in liposomes or microemulsions. The progestagen by encapsulating the progestagen may be encapsulated in the liposomes, thereby creating a delivery vehicle with a consistent absorption rate. Microemulsions may also be used as a delivery vehicle for progesterone. See Paul, et al, Curr. Sci., Apr. 25, 2001, 80(8):990-1001, which is herein incorporated by reference in its entirety including any references cited therein.

Without being limited to any particular theory, it is believed that the topical application of the progestagen composition to the palpebral part of the eye permits transdermal delivery of the active ingredient to the areas affected by dry eye diseases, such as but not limited to the lacrimal gland and accessory lacrimal glands. Further, the progestagen may act upon the progesterone receptors located in the lacrimal gland and lacrimal accessory glands, as well as other areas of the eye. Additionally, the progestagen may reduce viable T-cells due to apoptosis which in turn decreases the inflammatory state of the ocular surface and/or eyelids.

Typically, patients experience an improvement of their dry eye symptoms within about 3-7 days of initiation of treatment, and achieve a steady state within about 7 days.

### EXAMPLE 1

Twenty-three (23) patients with dry eye symptoms were tested using the Tear Break-up Time Test and Schirmer Test with anesthetic to determine the effectiveness of a progesterone composition. The patients also completed the OSDI questionnaire to assess the patient's perception of dry eye severity. The intraocular pressure for each patient was also determined before and after application of the progesterone composition. The progesterone composition was 15% progesterone in Vanicream®.

Each patient was instructed to cleanse their eyelids prior to applying the progesterone composition. A small amount of the cream, about 50 mg to about 100 mg, was applied to the upper and lower eyelids for each eye until the cream was no longer visible. The cream was applied twice a day, once in the morning and once at bedtime.

The average baseline testing scores were as follows:

| Test Performed | Average Score |
|---|---|
| Tear Break-up Test* | 5.64 |
| Schirmer Test* | 10.53 |
| Intraocular Pressure* | 14.06 |
| OSDI | 30.4 |

*The scores for the left and right eyes were averaged to obtain one value for each patient.

The testing scores after three weeks of treatment were as follows:

| Test Performed | Average Score |
|---|---|
| Tear Break-up Test* | 7.33 |
| Schirmer Test* | 13.78 |
| Intraocular Pressure* | 14.08 |
| OSDI | 25.23 |

*The scores for the left and right eyes were averaged to obtain one value for each patient.

The TBUT test showed a significant improvement after three weeks of treatment with a p-value of 0.084. The Schirmer Test showed a significant improvement after three weeks of treatment with a p-value of 0.151. The Intraocular Pressure Test showed no change in intraocular pressure. Patients reported a perceived improvement in their dry eye symptoms after use of the progesterone cream, with a p-value of 0.3 associated with a 20% improvement in symptoms after three weeks of treatment.

None of the patients reported any side effects from use of the progesterone cream and no allergic reactions were reported.

Although the present invention has been described in terms of specific embodiments, changes and modifications can be made out without departing from the scope of the invention which is intended to be defined only by the scope of the claims.

1. A composition for treating dry eye, comprising:
a therapeutically effective amount of a progestagen; and
a pharmaceutically acceptable carrier, wherein the composition is applied to a palpebral part of an eye.

2. The composition of claim 1, wherein the progestagen is selected from the group consisting of progesterone, a derivative of progesterone, synthetic progestagen, medroxyprogesterone acetate, norethindrone, norethindrone acetate, megestrol acetate, 17-a-hydroxyprogesterone caproate, and norgestrel.

3-13. (canceled)

14. The composition of claim 1, wherein the progestagen is present in a concentration from about 2% to about 30%.

15-73. (canceled)

75. The method of claim 74, wherein the progestagen is progesterone.

76. The method of claim 74, wherein the progestagen is a derivative of progesterone.

77. The method of claim 74, wherein the progestagen is a synthetic progestagen.

78. The method of claim 74, wherein the progestagen is medroxyprogesterone acetate.

79. The method of claim 74, wherein the progestagen is norethindrone.

80. The method of claim 74, wherein the progestagen is norethindrone acetate.

81. The method of claim 74, wherein the progestagen is megestrol acetate.

82. The method of claim 74, wherein the progestagen is 17-a-hydroxyprogesterone caproate.

83. The method of claim 74, wherein the progestagen is norgestrel.

84. The method of claim 74, wherein the composition is applied in an amount between about 25 mg and about 500 mg.

85. The method of claim 74, wherein the composition is applied in an amount between about 100 mg and about 400 mg.

86. The method of claim 74, wherein the composition is applied in an amount of about 160 mg.

87. The method of claim 74, wherein the progestagen is present in a concentration from about 2% to about 30%.

88. The method of claim 74, wherein the progestagen is present in a concentration from about 10% to about 30%.

89. The method of claim 74, wherein the progestagen is present in a concentration from about 15% to about 25%.

90. The method of claim 74, wherein the progestagen is present in a concentration of about 15%.

91. The method of claim 75, wherein the progesterone is present in a concentration from about 2% to about 30%.

92. The method of claim 75, wherein the progesterone is present in a concentration from about 10% to about 30%.

93. The method of claim 75, wherein the progesterone is present in a concentration from about 15% to about 25%.

94-96. (canceled)

97. The method of claim 74, wherein about 50mg to about 100mg of the composition is applied to each eye.

98. The method of claim 74, wherein about 80 mg of the composition is applied to each eye.

99-146. (canceled)
